(19) United States
(12) Patent Application Publication
(10) Pub. No.: US 2008/0254117 A1
(43) Pub. Date: Oct. 16, 2008

(54) PROCESS FOR PREPARING PRAMIPEXOLE DIHYDROCHLORIDE TABLETS

(76) Inventor: Noel Cotton, Columbus, OH (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 11/733,602

(22) Filed: Apr. 10, 2007

(51) Int. Cl. A61K 9/20; A61K 31/428

(52) U.S. Cl. 424/464; 514/367

(57) ABSTRACT

The present invention relates to a process for preparing tablets of pramipexole dihydrochloride. In particular, the present invention relates to a process for preparing tablets of pramipexole dihydrochloride wherein the tablets exhibit enhanced storage stability properties.

### FIELD OF INVENTION

The present invention relates to a process for preparing tablets of pramipexole dihydrochloride. In particular, the present invention relates to a process for preparing tablets of pramipexole dihydrochloride wherein the tablets exhibit enhanced storage stability properties.

### BACKGROUND OF THE INVENTION

Pramipexole is a known dopamine D2 receptor agonist. It is structurally different from the ergot-derived drugs, e.g., bromocriptine or pergolide. It is also pharmacologically unique in that it is a full agonist and has receptor selectivity for the dopamine D2 family of dopamine receptors. Pramipexole was originally disclosed in U.S. Pat. Nos. 4,731,374, 4,843,086 and 4,886,812, all of which are incorporated herein by reference.

Pramipexole is designated chemically as (S)-2-amino-4,5,6,7-tetrahydro-6-(propylamino)benzothiazole and has the molecular formula C₁₀H₁₇N₃S and a relative molecular mass of 211.33. The chemical formula is as follows:

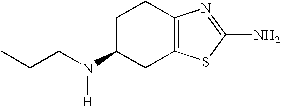

The salt form commonly used is pramipexole dihydrochloride monohydrate (molecular formula C₁₀H₂₁Cl₂N₃OS; relative molecular mass 302.27). Pramipexole dihydrochloride monohydrate is a white to off-white, tasteless, crystalline powder. Melting occurs in the range of 296° C. to 301° C., with decomposition. Pramipexole is a chiral compound with one chiral center. The pure (S)-enantiomer is obtained from the synthetic process by chiral recrystallization of one of the intermediates during synthesis.

Pramipexole dihydrochloride monohydrate is a highly soluble compound. Water solubility is more than 20 mg/mL and solubility in buffer media is generally above 10 mg/mL between pH 2 and pH 7.4. Pramipexole dihydrochloride monohydrate is not hygroscopic, and has a highly crystalline nature. Under milling, the crystal modification (monohydrate) does not change. Pramipexole is very stable in the solid state, yet in solution it is light sensitive.

Pramipexole immediate release (IR) tablets were first authorized in the USA in 1997, followed over the course of the next few years by marketing authorizations in the European Union (EU), Switzerland, Canada, and South America as well as in countries in Eastern Europe, the Near East, and Asia.

Pramipexole IR tablets are indicated in the EU and US for the treatment of signs and symptoms of either early Parkinson's Disease or advanced Parkinson's Disease in combination with levodopa. The IR tablets are indicated to be taken 3 times a day.

The manufacturing process for pramipexole dihydrochloride monohydrate tablets, currently marketed under the brand names MIRAPEX® and SIFROL®, results in a tablet which has a relatively stable shelf life wherein approximately 95% of the labeled amount of the active ingredient remains in the tablet after 18 months of storage. However, it is desirable to develop products having as close to zero degradation as possible upon being stored for extended periods of time.

The present invention relates to a process for preparing tablets of pramipexole dihydrochloride monohydrate wherein the tablets exhibit enhanced storage stability properties when compared to prior commercial formulations.

### SUMMARY OF THE INVENTION

For purposes of this disclosure and invention, hereinafter the term “pramipexole dihydrochloride” means pramipexole dihydrochloride and the pharmaceutically acceptable salts thereof including the monohydrate salt of pramipexole dihydrochloride.

In accordance with the present invention, there is provided a process for producing tablets of pramipexole dihydrochloride wherein the tablets exhibit enhanced storage stability properties when compared to prior commercial formulations. Compared to commercial formulations, the pramipexole dihydrochloride tablets produced in accordance with the process of the invention exhibit a higher percentage of active ingredient remaining when stored under conventional storage conditions along with a decreased amount of degradation products.

Further provided is a process for preparing tablets of pramipexole dihydrochloride wherein the process involves formulating tablets comprising intra-granular tableting ingredients, pramipexole dihydrochloride, a binder and extra-granular tableting agents. The process comprises the steps of sizing the intra-granular tableting ingredients to form substantially uniform sized particles of intra-granular tableting ingredients, forming a premix comprising the substantially uniform sized intra-granular tableting ingredients, the pramipexole dihydrochloride and the binder, granulating the premix and drying said granulated premix to an endpoint moisture content of from about 1.5% to about 2.5% to form a dried premix, mixing the extra-granular tableting agents with the dried premix to form a final blend and compressing the final blend into tablets.

Still further provided is a process for preparing pramipexole dihydrochloride tablets comprising intra-granular tableting ingredients, pramipexole dihydrochloride, a binder suspension and extra-granular tableting agents, wherein at least a portion of the process is performed in a closed system and comprises the steps of:

(a) sizing the intra-granular tableting ingredients to form substantially uniform sized particles of intra-granular tableting ingredients,

(b) mixing the particles of intra-granular tableting ingredients in a granulator-mixer,

(c) dissolving the pramipexole dihydrochloride in water to form an aqueous pramipexole dihydrochloride solution and adding the pramipexole dihydrochloride solution to the particles of intra-granular tableting ingredients in the granulator-mixer,

(d) preparing a binder suspension and adding the binder suspension to the mixer,

(e) mixing the particles of intra-granular tableting ingredients, pramipexole dihydrochloride solution and binder suspension in the granulator-mixer to form a premix,

(f) granulating said premix to form a granulated premix,

(g) drying said granulated premix to an endpoint moisture content of from about 1.5% to about 2.5%,

(h) mixing said granulated premix of step (g) with the extra-granular tableting agents and blending to form a final blend,

(i) compressing the final blend into tablets using a tablet press.

The tablets produced in accordance with the aforementioned process exhibit enhanced storage stability attributes when compared to commercial formulations.

A further aspect of the invention includes a pharmaceutical tablet formulation comprising pramipexole dihydrochloride, wherein the average amount of pramipexole dihydrochloride remaining in the tablet at 18 months under storage conditions of 25° C. and a relative humidity of 60% is at least about 97% of the labeled amount.

Another aspect of the invention includes a pharmaceutical tablet formulation comprising pramipexole dihydrochloride, wherein the average amount of pramipexole dihydrochloride remaining in the tablet at 24 months under storage conditions of 25° C. and a relative humidity of 60% is at least about 95% of the labeled amount and further may be, preferably, at least about 97%.

An additional aspect of the invention includes a pharmaceutical tablet formulation comprising pramipexole dihydrochloride, wherein the average amount of total degradation products present in the tablet at 18 months under storage conditions of 25° C. and a relative humidity of 60% is less than about 1.0%.

The term “average amount” as used herein is calculated by determining the amount of the designated product (either active ingredient or degradation product) present in a particular sample of product and then taking an average of the samples of product.

The term “sample of product” included 21 count bottles, 90 count bottles and blister packs of the tablets.

These and other features, benefits and advantages of the invention will be apparent from the following disclosure.

### BRIEF DESCRIPTION OF THE DRAWINGS

FIG. 1 is a flow chart showing a process for producing pramipexole dihydrochloride tablets according to one aspect of the invention.

FIG. 2 is a graphical depiction comparing storage stability of tablets prepared according to the invention compared to a commercial formulation.

### DETAILED DESCRIPTION OF THE INVENTION

According to the present invention, pramipexole dihydrochloride tablets can be prepared which exhibit enhanced storage stability over known commercial formulations. This is valuable in the pharmaceutical arena as it enables pharmaceutical manufacturers to produce and store the pramipexole dihydrochloride tablets for longer periods thereby reducing concern as to whether the product has exceeded its useful life and requires disposal. This, in turn, enables pharmacies, and ultimately consumers, to enjoy the benefits of reduced costs associated with the need to monitor the efficacy of a product and the need to replenish the market supply due to expiration of the product.

In accordance with the invention, it has been found that by controlling certain parameters during the manufacture of pramipexole dihydrochloride tablets, the resulting tablets exhibit enhanced stability when compared to commercial formulations. In particular, controlling the particle size of intra-granular tableting ingredients so that they possess a relative substantial uniformity, preparation and use of a binder suspension, performing the process in a closed system, as well as controlling the moisture content of the product prior to tableting enables the production of a pramipexole dihydrochloride tablet which has highly desirable storage stabilty enhancements over known formulations.

In accordance with the above, the pramipexole dihydrochloride tablets of the invention comprise intra-granular tableting ingredients, pramipexole dihydrochloride, a binder and extra-granular tableting agents. The process of the invention comprises the steps of sizing the intra-granular tableting ingredients to form substantially uniform sized particles of intra-granular tableting ingredients, forming a premix comprising the uniformly sized intra-granular tableting ingredients, the pramipexole dihydrochloride and the binder, granulating the premix and drying said granulated premix to an endpoint moisture content (Loss on Drying (LOD) at 95° C.) of from about 1.5% to about 2.5% to form a dried premix, mixing the extra-granular tableting agents with the dried premix to form a final blend and compressing the final blend into tablets.

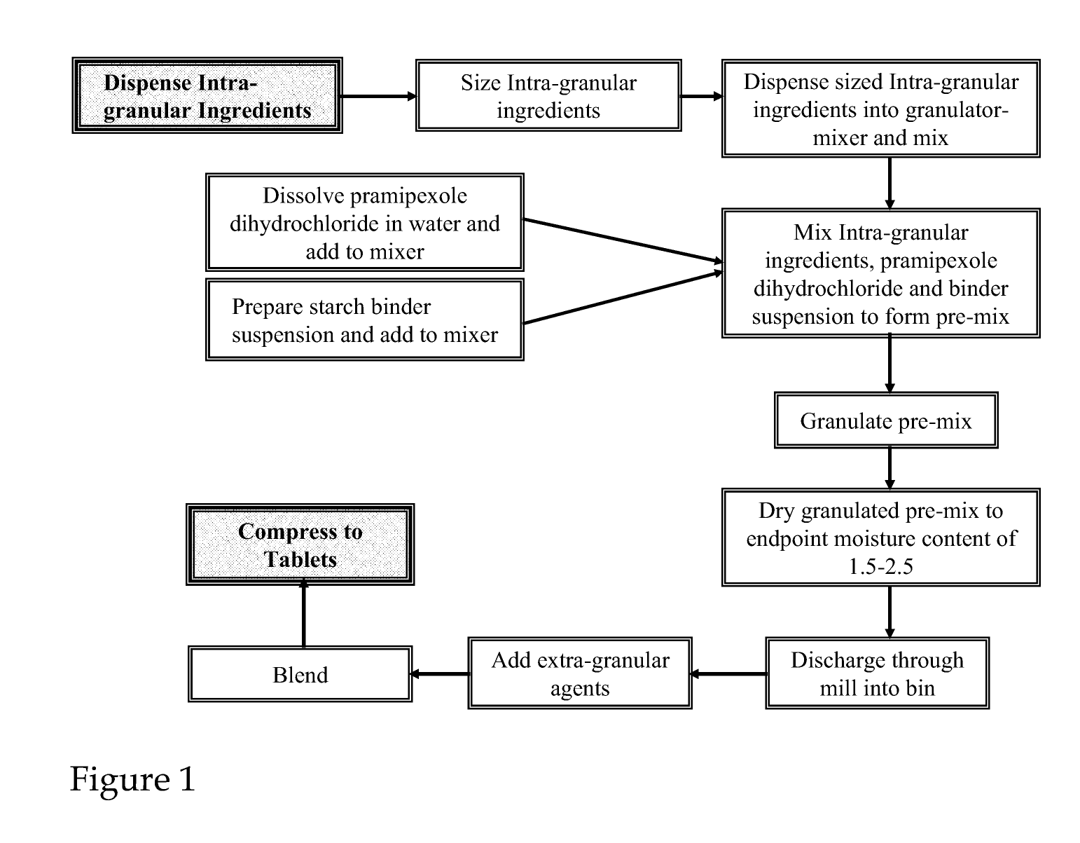

More particularly, one embodiment of the process of the invention is substantially as shown in FIG. 1. The process shown in FIG. 1 involves a process for preparing pramipexole dihydrochloride tablets comprising intra-granular tableting ingredients, pramipexole dihydrochloride, a binder and extra-granular tableting agents, wherein at least a portion of the process is performed in a closed system. The process comprises the steps of:
(a) sizing the intra-granular tableting ingredients to form substantially uniform sized particles of intra-granular tableting ingredients,
(b) mixing the particles of intra-granular tableting ingredients in a granulator-mixer,
(c) dissolving the pramipexole dihydrochloride in water to form an aqueous pramipexole dihydrochloride solution and adding the pramipexole dihydrochloride solution to the particles of intra-granular tableting ingredients in the granulator-mixer,
(d) preparing a binder suspension and adding the binder suspension to the granulator-mixer,
(e) mixing the particles of intra-granular tableting ingredients, pramipexole dihydrochloride solution and binder suspension in the granulator-mixer to form a premix,
(f) granulating said premix to form a granulated premix,
(g) drying said granulated premix to an endpoint moisture content of from about 1.5% to about 2.5%,
(h) mixing said granulated premix of step (g) with the extra-granular tableting agents and blending to form a final blend,
(i) compressing the final blend into tablets using a tablet press.

According to the process of FIG. 1, the sizing step (a) can be accomplished by using a conventional particle sizing apparatus such as a comil. Initially, the intra-granular particles are sized such that they exhibit a substantial uniformity. More specifically, the particles are sized such that they pass through a 1.4 mm screen prior to addition to the granulator-mixer.

The sized intra-granular tableting ingredients from step (a) are then transferred from the into a high shear granulator-mixer and mixed together. After mixing, an aqueous solution of pramipexole dihydrochloride is made. The aqueous solution of pramipexole dihydrochloride is then added to the mixture of intra-granular ingredients and mixed therewith.

Separately, a starch based binder is prepared. The starch based binder for this process is prepared such that it forms a suspension. In particular, the starch based binder suspension is formed by heating water in a jacketed tank to 75° C. (±2° C.) and adding corn starch NF which has been mixed with an equal amount of water at room temperature, to the heated water with mixing. The starch based suspension is then mixed for about 5 minutes and then allowed to cool to about 50° C. (±5° C.). The cooled starch based suspension is then added to the granulator-mixer containing the intra-granular ingredients and pramipexole dihydrochloride thereby forming a premix.

The premix is then granulated. The granulated premix is then transferred to a fluid bed and dried to an endpoint moisture content (Loss On Drying (LOD) at 95° C.) of from about 1.5% to about 2.5%. The moisture content is measured with a moisture analyzer such as, for example, a Mettler Toledo Moisture Analyzer. The dried, granulated mixture is then transferred to a bin through a mill having a 1.4 mm screen.

The extra-granular tableting ingredients are then passed through a comil having a screen size of 1.4 mm, and into a bin. The extragranular ingredients are then added to the dried, granulated mixture containing the pramipexole dihydrochloride and intra-granular ingredients to form a final blend which is blended on a tumbler. The final blend is tested for uniformity after approximately 200 revolutions on the tumbler. Once sample uniformity is achieved (average of 10 samples is 90-110% of the labeled claim amount with an RSD of no more than 5.0% for all ten samples), the final blend is then dispensed into a tablet press, such as, for example, a Fette Press, model 2090i or 2090 IC single rotary, and the tablets are compressed to the desired size such that the desired dosage is achieved.

The acceptable criteria for uniformity of dosage of the tablets for pramipexole dihydrochloride tablets is set forth in USP <905>.

The foregoing process is preferably conducted in a closed system. Specifically, once the initial ingredients are added to the system (intra-granular tableting ingredients, pramipexole dihydrochloride, a binder and extra-granular tableting agents), their exposure to the atmosphere is reduced as much as practicable. Accordingly, the system is designed to reduce exposure of the ingredients to atmospheric conditions by connecting the components thereof such that minimal exposure to the atmosphere outside the system is achieved. This is done to reduce the chance of exposure to excess atmospheric moisture and light, for example, which could adversely affect the desired stability properties of the end product.

According to a further aspect of the invention, the intra-granular tableting ingredients, pramipexole dihydrochloride, and binder suspension are divided into at least two batches before processing. Once processed to the granulated premix stage, the separate batches are combined and then formulated with the extra-granular tableting agents prior to formulating into the final blend.

The intra-granular tableting ingredients include mannitol-D USP, colloidal silicon dioxide NF, povidone (K25) USP, corn starch NF and purified water USP.

The mannitol-D used in the process of the present invention is a modification product of mannitol having a beta content of not more than 10%.

The extra-granular tableting agents of the present invention include colloidal silicon dioxide NF, corn starch NF and magnesium stearate NF.

With respect to the intra-granular tableting ingredients and extra granular tableting ingredients, the following table represents the preferred amounts oftableting ingredients in each tablet as a percentage of the overall amount used in each batch as well as the amount of API (pramipexole dihydrochloride):

TABLE 1

| Ingredient | % per batch |
|---|---|
| Mannitol-D | 50-60 |
| Corn Starch | 35-45 |
| Colloidal Silicon Dioxide | 1-3 |
| Povidone | 1-3 |
| Magnesium Stearate | 1-3 |
| API | ** |

**The amount of API is dependent upon the desired tablet strength.

Tablet strengths can be from 0.125 mg to 1.5 mg with typical strengths being 0.125 mg, 0.25 mg, 0.50 mg, 0.75 mg, 1 mg and 1.5 mg.

The following table represents one formulation example of a 0.75 mg tablet according to the present invention:

TABLE 2

| | |
|---|---|
| ACTIVE INGREDIENT (API) | mg/tablet |

Strength 0.75 mg

| | |
|---|---|
| pramipexole dihydrochloride | 0.750 |

INTRAGRANULAR TABLETING INGREDIENTS

| | |
|---|---|
| Mannitol-D USP | 183.0 |
| Colloidal silicon dioxide NF | 1.8 |
| Povidone (K25) NF | 3.45 |
| Corn Starch NF | 92.7 |

| | |
|---|---|
| Water, purified NF | QS |

EXTRA-GRANULAR INGREDIENTS

| | | |
|---|---|---|
| Colloidal silicon dioxide NF | 1.8 | |
| Corn starch NF | 27.0 | |
| Magnesium stearate NF | 4.5 | |
| Theoretical total weight | 315 | mg |

The advantages to be realized from using the processes of the invention to produce the pramipexole dihydrochloride tablets of the invention include enhanced storage stability properties. Such enhanced storage stability properties include, but are not necessarily limited to, enhanced shelf life and decreased degradation products.

The enhanced shelf life of the pramipexole dihydrochloride tablets prepared according to the processes of the present invention is exhibited by the ability of the tablets to retain a higher percentage of active ingredient when stored under certain conditions compared to commercial formulations stored under the same conditions.

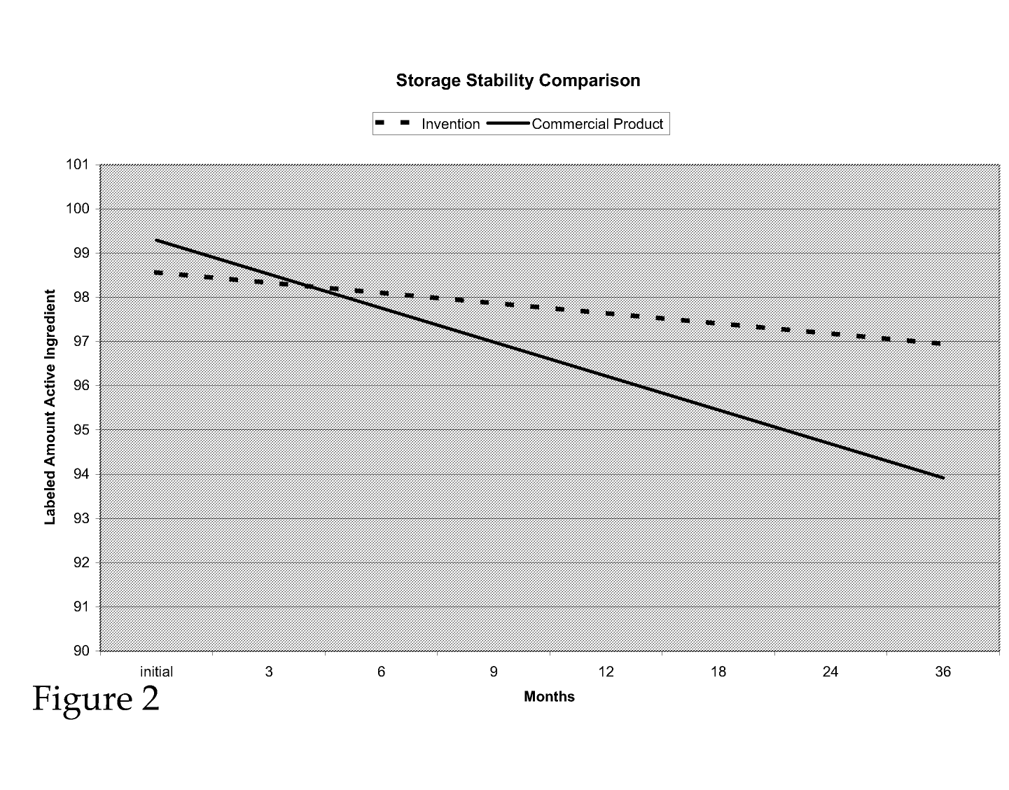

In particular, the pramipexole dihydrochloride tablets prepared according to the process of the present invention has an average amount of pramipexole dihydrochloride remaining in the tablet at 18 months under storage conditions of 25° C. and a relative humidity of 60% of at least about 97% of the labeled amount. Commercial formulations stored under the same conditions average less than 95.8% of the labeled amount. As shown in FIG. 2, the trend for the amount of active ingredient present in the stored tablets prepared according to the invention can be projected out to 24 and even 36 months where even at 36 months greater than 95% of the labeled amount should remain. This can be compared to the current commercial formulation where the amount of active ingredient remaining in the stored tablets drops below 95% prior to the 24 month period. This of course is significant as is allows for longer shelf life of the product and thus cost savings to consumers as the product does not have to be replaced by the manufacturer as frequently due to expiration of the unused product stored by the manufacturer, distributor and/or pharmacist.

A further advantage of the pramipexole dihydrochloride tablets prepared according to the process of the present invention involves the decreased amounts of degradation products which appear in the tablets upon storage.

Upon testing samples of tablets prepared according to the present invention and commercial formulations of pramipexole dihydrochloride when stored for 18 months under storage conditions of 25° C. and a relative humidity of 60%, the following represents the amount of the above total degradation products detected in the samples:

TABLE 3
Total Degradation Product (%)

| Product | Invention | Commercial |
|---|---|---|
| 0.125 mg Tablet/21 count bottle | 1.5 | N/A |
| 0.125 mg Tablet/90 count bottle | 1.2 | N/A |
| 0.5 mg Tablet/90 count bottle | 0.8 | 2.4 |
| 1.0 mg Tablet/blister pack | 0.6 | 1.9 |
| 1.0 mg Tablet/90 count bottle | 0.8 | 1.35 (avg. of 8 samples) |
| 1.5 mg Tablet/90 count bottle | 0.6 | 1.1 (avg. of 5 samples) |
| 1.5 mg Tablet/blister pack | 0.4 | 2.1 (avg. of 2 samples) |
| Average Total Degradation | 0.84 | 1.45 (17 samples) |
| Product | (7 samples) | |

The following Examples are representative of the process used to prepare pramipexole dihydrate tablets according to the invention.

### EXAMPLE 1

The following example describes a process of the invention as used to prepare 0.125 mg tablets of pramipexole dihydrochloride:

In a 600 L bin, the following ingredients were added by passing them through a Comil (model 194) equipped with a 1.4 mm screen at a setting of 900 RPM:

| | | |
|---|---|---|
| Mannitol D: | 98.910 | kg |
| Colloidal Silicon Dioxide NF (Aerosil 200): | 940 | g |
| Povidone, USP (Kollidon K-25): | 1.880 | kg |
| Corn Starch NF: | 48.380 | kg |

A jacketed tank was heated to 75° C. (±2° C.) with 17.609 kg purified water. In a separate tank, 2.891 kg corn starch NF was added to 3.0 kg purified water with mixing for a minimum of about 2 minutes at a rate of about 300 RPM thereby forming a paste. The paste was then added to the water in the tank which has been heated to 75° C. and mixed for a minimum of 5 minutes at a rate of about 300 RPM forming a starch suspension. The starch suspension was then cooled to about 55° C. (±5° C.). In a separate tank, 250 g pramipexole dihydrochloride monohydrate was added to 8.980 kg purified water with mixing and mixed at about 200 RPM for a minimum of 2 minutes. A granulator (PowRex VG-600) was then charged with the comilled mannitol, colloidal silicon dioxide, povidone, and corn starch mixture upon reaching an air inlet temperature of about 85° C. and mixed (main blade 160 RPM/Cross screw 1760 RPM) for about 2 minutes. The pramipexole solution was then added to the granulator and mixed for about 1 minute. The tank holding the pramipexole was then rinsed with 1.5 kg purified water and the rinse was added to the granulator and mixed for an additional minute. The starch suspension was then added to the granulator and mixed for about 1 minute. The speed of the mixer was then increased (main blade 200 RPM/Cross screw 2460 RPM) and the mixture was mixed for an additional 3 minutes with scraping at the 2 minute mark. The mixture was then transferred to a Glatt Fluid Bed and dried to a target endpoint moisture content (LOD) of from about 1.5% to about 2.5%. The batch was then discharged into a separate bin throught a glatt mill having a screen size of about 1.4 mm. The above procedure was then repeated and a second identical batch was made and added to the bin containing the first batch.

The following ingredients were passed through a Comil (Model 194 Quadro) having a 1.4 mm screen and into the bin containing the previously granulated materials:

| | |
|---|---|
| Colloidal silicon dioxide NF (Aerosil 200): | 1.880 kg |
| Corn Starch NF: | 29.200 kg |
| Magnesium stearate NF: | 4.920 kg |

The above mixture was then placed on a tumbler and blended at about 7 RPM for about 200 revolutions to form a final blend. The final blend is then compressed on a tablet press and pramipexole dihydrochloride tablets having the appropriate dosage amount were formed.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A process for preparing pramipexole dihydrochloride tablets comprising intra-granular tableting ingredients, pramipexole dihydrochloride or a pharmaceutically acceptable salt thereof, a binder suspension and extra-granular tableting agents, wherein at least a portion of the process is performed in a closed system and comprises the steps of:
(a) sizing the intra-granular tableting ingredients to form uniformly sized particles of intra-granular tableting ingredients,
(b) mixing the particles of intra-granular tableting ingredients in a granulator-mixer,
(c) dissolving the pramipexole dihydrochloride or a pharmaceutically acceptable salt thereof, in water to form an aqueous pramipexole dihydrochloride solution and adding the pramipexole dihydrochloride solution to the particles of intra-granular tableting ingredients in the granulator-mixer,
(d) preparing a binder ssuspension and adding the binder suspension to the granulator-mixer,
(e) mixing the particles of intra-granular tableting ingredients, pramipexole dihydrochloride solution and binder suspension in the granulator-mixer to form a premix,
(f) granulating said premix to form a granulated premix,
(g) drying said granulated premix to an endpoint moisture content of from about 1.5% to about 2.5,
(h) mixing said granulated premix of step (g) with the extra-granular tableting agents and blending to form a final blend,
(i) compressing the final blend into tablets using a tablet press.

2. The process of claim 1 wherein the process steps (a)-(g) are preformed in at least two separate batches using a portion of the total amount required for the total batch for each of the separate batches, further wherein the two batches are combined after the drying step (g).

3. The process of claim 1 wherein pramipexole dihydrochloride monohydrate salt is used.

4. The process of claim 1 wherein the sizing of step (a) is performed by milling the ingredients so that they pass through a 1.4 mm screen.

5. The process of claim 1 wherein the drying of the granulated premix in step (g) is by fluid bed.

6. The process of claim 1 wherein the binder suspension is an aqueous suspension comprising corn starch.

7. The process of claim 1 wherein the intra-granular tableting ingredients comprise mannitol-D, colloidal silicone dioxide, povidone and corn starch.

8. The process of claim 1 wherein the extra-granular tableting agents comprise colloidal silicon dioxide, starch and magnesium stearate.

9. The process of claim 7 wherein the mannitol-D has no more than 10% beta modification product present.
